(19) United States
(12) Patent Application Publication
(10) Pub. No.: US 2025/0198930 A1
(43) Pub. Date: Jun. 19, 2025

(54) MULTIFUNCTIONAL COMPOSITE BIOPROBE AND PREPARATION METHOD AND USE THEREOF

(71) Applicants: NINGBO INSTITUTE OF MATERIALS TECHNOLOGY AND ENGINEERING, CHINESE ACADEMY OF SCIENCES, Ningbo (CN); NINGBO CIXI INSTITUTE OF BIOMEDICAL ENGINEERING, Ningbo (CN); ZHEJIANG CANCER HOSPITAL, Hangzhou (CN)

(72) Inventors: Aiguo WU, Ningbo (CN); Jie LIN, Ningbo (CN); Xiaoxia WU, Ningbo (CN); Guoliang SHAO, Ningbo (CN); Dinghu ZHANG, Ningbo (CN)

(21) Appl. No.: 18/845,305

(22) PCT Filed: Mar. 17, 2023
(86) PCT No.: PCT/CN2023/082086

§ 371 Date: Sep. 9, 2024

(30) Foreign Application Priority Data
Dec. 13, 2022 (CN) 202211593925.2

(51) Int. Cl. G01N 21/64; B82Y 15/00; G01N 21/65

(52) U.S. Cl. CPC G01N 21/6486; G01N 21/658; B82Y 15/00

(57) ABSTRACT

The present disclosure provides a multifunctional composite bioprobe and a preparation method and use thereof. The multifunctional composite bioprobe includes one or more of noble metal SERS bioprobes and one or more of magnetic semiconductor SERS bioprobes. According to the present disclosure, the functional superposition of the noble metal SERS bioprobe and the magnetic semiconductor SERS bioprobe is realized through the simplest and most effective way, which improves the sensitivity and accuracy of the detection method, and endows magnetic enrichment characteristics for the detection method.

### TECHNICAL FIELD

The present disclosure belongs to the technical field of life sciences, and relates to a multifunctional composite bioprobe and a preparation method and use thereof.

### BACKGROUND

Surface enhanced Raman scattering (SERS) is based on the localized plasma resonance of the nanostructure of noble metals or metal compounds, which significantly enhances the Raman signal by 10⁶-10¹⁰ times. SERS spectroscopy technology has the advantages of good selectivity, high sensitivity, no photobleaching, anti-interference, rapidness and non-destruction, etc. Surface enhanced Raman scattering spectroscopy has shown great application potential in the field of optical detection, such as in vitro diagnosis and liquid biopsy. At present, conventional SERS bioprobes are divided into noble metal probes and semiconductor bioprobes. Noble metal probes have the advantage of high detection sensitivity, and semiconductor bioprobes have the functional advantages, such as good signal stability and photoelectromagnetic response. Usually, in order to combine their advantages, noble metals and functional semiconductor materials are compounded by chemical synthesis. This method is relatively complicated, and it is not easy to obtain composite materials with good uniformity, which seriously restricts the application and development of noble metals and semiconductor materials in the field of SERS.

### SUMMARY

In view of the drawbacks of the prior art, the present disclosure provides a multifunctional composite bioprobe, a preparation method and use thereof.

An object of the present disclosure is achieved by the following technical solutions:

A multifunctional composite bioprobe includes one or more of noble metal SERS bioprobes and one or more of magnetic semiconductor SERS bioprobes.

Preferably, the particle size of the noble metal SERS bioprobe is 0.1 nm-10,000 nm; more preferably, the particle size is 0.1-1000 nm, still more preferably, the particle size is 0.1-800 nm, and still more preferably, the particle size is 1-500 nm.

Preferably, the particle size of the magnetic semiconductor SERS bioprobe is 0.1 nm-10,000 nm; more preferably, the particle size is 0.1-1,000 nm, still more preferably, the particle size is 0.1-800 nm, and still more preferably, the particle size is 1-500 nm.

Preferably, the morphology of the noble metal SERS bioprobe and the magnetic semiconductor SERS bioprobe includes but is not limited to any one of lamellar, tetrahedral, hexahedral, octahedral, dodecahedral, hollow cage, round particle, and rod shapes.

Preferably, the noble metal SERS bioprobe includes a noble metal material with SERS performance, the noble metal material includes but is not limited to one or more of single-substance materials such as gold, silver, palladium, copper, and a composite material, and the composite material is a composite material containing gold, silver, palladium or copper.

Preferably, the particle size of the noble metal material is 0.1-500 nm, and optionally, the particle size of the noble metal material is any one of 0.5, 1, 5, 10, 20, 30, 40, 50, 100, 150, 200, 250, 300, 350, 400, 450, 500, or a range value between any two values.

Preferably, the magnetic semiconductor SERS bioprobe includes a magnetic metal oxide with SERS performance, and the magnetic metal oxide includes but is not limited to an oxide material containing one or more of Fe, Zn, Co, Ni, Cr, and Mn.

Preferably, the particle size of the magnetic metal oxide is 0.1-500 nm. Optionally, the particle size of the magnetic metal oxide is any one of 0.5, 1, 5, 10, 20, 30, 40, 50, 100, 150, 200, 250, 300, 350, 400, 450, 500 or a range between any two values. More preferably, the particle size is 1-50 nm.

Preferably, the magnetic metal oxide is an oxide material formed by Fe and one or more of Zn, Co, Ni, Cr, and Mn. The magnetic semiconductor SERS bioprobe constructed based on the magnetic metal oxide formed by doping Fe oxide with one or more of Zn, Co, Ni, Cr, and Mn has a more excellent SERS enhancement effect, which is conducive to improving the detection sensitivity and accuracy.

Preferably, the magnetic metal oxide is ZnₓFe₃₋ₓO₄, 0<x<3.

Preferably, a method for preparing the magnetic metal oxide includes the following steps: mixing a solution of one or more of a zinc salt, a cobalt salt, a nickel salt, a chromium salt, a manganese salt and an iron salt with a solution formed by an alkaline inorganic matter, an organic acid with long alkyl chain and a polar organic solvent, reacting to obtain initial magnetic particles, and performing phase transfer to obtain magnetic metal oxide nanoparticles. There are a number of methods for preparing magnetic metal oxides, but the magnetic metal oxide nanoparticles prepared by many preparation methods do not have SERS performance. The present disclosure has shown that only the magnetic metal oxide nanoparticles prepared by the method of the present disclosure have SERS performance.

Iron salts include but are not limited to one or more of ferric chloride, ferrous chloride, ferric sulfate, ferrous sulfate, ammonium ferrous sulfate, ferric nitrate, and ferrous nitrate; zinc salts include but are not limited to one or more of zinc chloride, zinc sulfate, and zinc nitrate; cobalt salts include but are not limited to one or more of cobalt chloride, cobalt sulfate, and cobalt nitrate; nickel salts include but are not limited to one or more of nickel chloride, nickel sulfate, and nickel nitrate; chromium salts include but are not limited to one or more of chromium chloride, chromium sulfate, and chromium nitrate; manganese salts include but are not limited to one or more of manganese chloride, manganese sulfate, and manganese nitrate; alkaline inorganic matters include but are not limited to barium hydroxide, potassium hydroxide, calcium hydroxide, sodium hydroxide, and ammonium hydroxide, etc.; organic acids with long alkyl chain include but are not limited to oleic acid, octadecanoic acid, hexadecanoic acid, tetradecanoic acid, and dodecanoic acid, etc.; polar organic solvents include but are not limited to methanol, ethanol, isopropanol, and other highly polar organic solvents.

The solution of one or more of a zinc salt, a cobalt salt, a nickel salt, a chromium salt, a manganese salt and an iron salt is a solution formed by dissolving one or more of a zinc salt, a cobalt salt, a nickel salt, a chromium salt, a manganese salt and an iron salt in water; in the solution of one or more of a zinc salt, a cobalt salt, a nickel salt, a chromium salt, a manganese salt and an iron salt, the concentration of iron salt is 2-200 mmol/L.

The solution formed by an alkaline inorganic matter, an organic acid with long alkyl chain and a polar organic solvent is formed by adding an alkaline inorganic matter to an organic acid with long alkyl chain and a polar organic solvent. Each gram of an alkaline inorganic matter is added to 1-100 mL of an organic acid with long alkyl chain and 1-100 mL of a polar organic solvent.

Preferably, the volume ratio of the solution of one or more of a zinc salt, a cobalt salt, a nickel salt, a chromium salt, a manganese salt and an iron salt to the solution formed by an alkaline inorganic matter, an organic acid with long alkyl chain and a polar organic solvent is 1:10-10:1.

Preferably, the solution of one or more of a zinc salt, a cobalt salt, a nickel salt, a chromium salt, a manganese salt and an iron salt is mixed with the solution formed by an alkaline inorganic matter, an organic acid with long alkyl chain and a polar organic solvent to react; the reaction temperature is 100-500° C. and the reaction time is 1-50 h.

Preferably, the reaction time is any one of 1, 2, 3, 5, 8, 10 h or a range value between any two values. Optionally, the reaction temperature is any one of 100, 200, 250, 300, 350, 400, 450, 500° C. or a range value between any two values.

Preferably, the phase transfer includes the following steps: adding the initial magnetic particles and citric acid to the organic solvent, and stirring at room temperature for 1-50 h. Optionally, the stirring time is any one of 5, 8, 10, 15, 20 h or a range value between any two values. Examples of organic solvents include chloroform, N, N-dimethylformamide, chloroform, carbon tetrachloride, formamide, DMSO, tetrahydrofuran or pyridine, etc.

Through the phase transfer reaction, the nanoparticles are transferred from the oil phase to the water phase, which improves the dispersibility of the nanoparticles in water, improves or presents the SERS performance of the nanoparticles.

Preferably, the noble metal SERS bioprobe includes a noble metal material, Raman/fluorescent signal molecules, biomacromolecules and antibody targeting proteins in sequence from the inside to the outside; the magnetic semiconductor SERS bioprobe includes a magnetic metal oxide, Raman/fluorescence signal molecules, biomacromolecules and antibody targeting proteins in sequence from the inside to the outside.

Preferably, the Raman/fluorescent signal molecules are substances having both fluorescent and Raman properties, including but not limited to one or more of IR783, IR780, 3,3′-diethylthiotricarbocyanine iodide (DTTC), rhodamine, crystal violet, alizarin red, Nile blue, and methylene blue.

Preferably, the biomacromolecules include but are not limited to one or more of polydopamine, dopamine hydrochloride, bovine serum albumin, reduced bovine serum albumin, and polyethylene glycol. Any biomacromolecule with biomacromolecular characteristics can be the biomacromolecule of the present disclosure.

Preferably, the antibody targeting proteins are proteins and polypeptides targeting tumor markers. The tumor markers can be listed as tumor cells, protein markers, exosomes, CtDNA, etc., and the tumors can be listed as breast cancer, liver cancer, lung cancer, esophageal cancer, etc. The aforementioned proteins and polypeptides can be listed as folic acid antibody protein, Trop2 antibody protein and GE11 polypeptide, etc.; without limiting the aforementioned proteins and polypeptides, any one with tumor targeting characteristics can be an antibody targeting protein of the present disclosure.

The Raman/fluorescence signal molecules and biomacromolecules in the noble metal SERS bioprobe can be the same as or different from those in the magnetic semiconductor SERS bioprobe; the antibody targeting proteins in the two probes are the same substance, so that the noble metal SERS bioprobe and the magnetic semiconductor SERS bioprobe can target the same tumor marker.

Another object of the present disclosure is achieved by the following technical solutions:

A method for preparing the multifunctional composite bioprobe includes the following steps: mixing one or more of the noble metal SERS bioprobes with one or more of the magnetic semiconductor SERS bioprobes in a liquid or solid form to obtain the multifunctional composite bioprobe.

Still another object of the present disclosure is achieved by the following technical solutions:

Use of the multifunctional composite bioprobe in in-vitro testing includes the following steps: mixing one or more of the noble metal SERS bioprobes with one or more of the magnetic semiconductor SERS bioprobes in a liquid or solid form and then adding the mixture to a system to be tested; or adding one or more of the noble metal SERS bioprobes and one or more of the magnetic semiconductor SERS bioprobes to a system to be tested successively in a liquid or solid form.

Preferably, the use further includes the following steps: after adding the multifunctional composite bioprobe to the system to be tested and combining the multifunctional composite bioprobe with the target in the system to be tested, carrying out magnetic enrichment and separation, then performing Raman spectroscopy and/or fluorescence spectroscopy to determine the concentration of the target in the system to be tested.

The excitation light wavelength used in Raman spectroscopy and/or fluorescence spectroscopy is 266-1,064 nm; preferably, the lower limit of the excitation light wavelength is 266 nm, and the upper limit is selected from any one of 325, 488, 514, 532, 633, 647, 785, and 1,064 nm; preferably, the excitation light wavelength is selected from any one of 266 nm, 325 nm, 488 nm, 514 nm, 532 nm, 633 nm, 647 nm, 785 nm, and 1,064 nm.

Still another object of the present disclosure is achieved by the following technical solutions:

An in vitro detection device includes the multifunctional composite bioprobe. The in vitro detection device can be listed as a sensor, a detector, a spectral responder, etc.

Compared with the prior art, the present disclosure has the following beneficial effects:
(1) In the multifunctional composite bioprobe of the present disclosure, the noble metal SERS bioprobe has the characteristic of high detection sensitivity, and the magnetic semiconductor SERS bioprobe has the characteristic of magnetic enrichment. The noble metal SERS bioprobe and the magnetic semiconductor SERS bioprobe target tumor cells, and the rapid enrichment of nanoprobes and tumor cells can be achieved by the magnetic field, to facilitate to improve the detection sensitivity and accuracy of tumor cells;
(2) Both the noble metal SERS bioprobe and the magnetic semiconductor SERS bioprobe can provide Raman spectral signals and fluorescence spectral signals, and improve the accuracy of the detection system through the dual-signal molecular mode; meanwhile, the magnetic semiconductor SERS bioprobe also has the ability to provide magnetic resonance imaging. Therefore, the noble metal SERS bioprobe and the magnetic semiconductor SERS bioprobe are used in combination to realize Raman, fluorescence and nuclear magnetic resonance multimodal applications;
(3) The noble metal SERS bioprobe and the magnetic semiconductor SERS bioprobe target to identify the same tumor cell; the Raman spectroscopy and/or fluorescence spectroscopy show that two or more Raman and/or fluorescence signals appear simultaneously in the detected cells, which can ensure the e accuracy of tumor detection and eliminate the false positive interference of blood cells in peripheral blood samples;
(4) The noble metal SERS bioprobe and magnetic semiconductor SERS bioprobe provided by the present disclosure are added to a system to be tested for reaction in a simple mixing manner, which avoids the production of noble metal-semiconductor composite material by complex chemical synthesis, and has the characteristics of simple process, simplified equipment, low cost, safety and feasibility;
(5) In the present disclosure, the functional superposition of the noble metal SERS bioprobe and the magnetic semiconductor SERS bioprobe is realized through the simplest and most effective way, which improves the sensitivity and accuracy of the detection method, and endows magnetic enrichment characteristics for the detection method;
(6) The magnetic metal oxide in the magnetic semiconductor SERS bioprobe of the present disclosure is preferably an oxide material of Fe and one or more of Zn, Co, Ni, Cr, Mn, and has a better SERS enhancement effect.

### BRIEF DESCRIPTION OF THE DRAWINGS

FIG. 1 is a TEM spectrum of a noble metal gold nanomaterial prepared in Example 1;

FIG. 2 is a TEM spectrum of a noble metal gold nanomaterial prepared in Example 2;

FIG. 3 is a TEM spectrum of a noble metal gold nanomaterial prepared in Example 3;

FIG. 4 is a TEM spectrum of Zn_{0.2}Fe_{2.8}O₄ magnetic nanoparticles prepared in Example 4;

FIG. 5 is a TEM spectrum of Zn_{0.2}Fe_{2.8}O₄ magnetic nanoparticles prepared in Example 5;

FIG. 6 is a TEM spectrum of Zn_{0.2}Fe_{2.8}O₄ magnetic nanoparticles prepared in Example 6;

FIG. 7a is a surface enhanced Raman spectrum corresponding to 4MBA molecules on gold nanoparticles in Example 1, and FIG. 7b is a surface enhanced Raman spectrum corresponding to crystal violet molecules on Zn_{0.2}Fe_{2.8}O₄ magnetic nanoparticles in Example 5;

FIG. 8 (a) is a fluorescence spectrum of a luminescent indicator CCK-8 modified on the surface of magnetic nanoparticles in Example 5, and FIG. 8 (b) is a fluorescence spectrum of a luminescent indicator CCK-8 modified on the surface of noble metal gold nanomaterials in Example 1;

FIG. 9 is a SERS spectrum of magnetic nanoparticles in Example 5 and Fe₃O₄ magnetic nanoparticles in Comparative Example 1 relative to methylene blue molecules;

FIG. 10 is an enrichment and capture image of tumor cells by a Zn_{0.2}Fe_{2.8}O₄-Alizarin Red-PDA-Trop2 antibody protein nanoprobe in Example 8;

FIG. 11 is a SERS spectrum of an Au-IR783-rBSA-Trop2 antibody protein nanoprobe in Example 7 and a Zn_{0.2}Fe_{2.8}O₄-Alizarin Red-PDA-Trop2 antibody protein nanoprobe in Example 8 after binding to MCF7 breast cancer cells.

### DETAILED DESCRIPTION OF THE EMBODIMENTS

The technical solutions of the present disclosure will be further described below in conjunction with specific embodiments and accompanying drawings. It should be appreciated that the specific embodiments described herein are only used to help understand the present disclosure and are not intended for specific limitations of the present disclosure. The accompanying drawings used herein are only for better illustrating the disclosure of the present disclosure and do not have limitation on the scope of the protection. Unless otherwise specified, all raw materials used in the embodiments of the present disclosure are commonly used raw materials in the art, and all methods used in the embodiments are conventional methods in the art.

### Example 1

### Preparation of Noble Metal Gold Nanomaterials (3 nm)

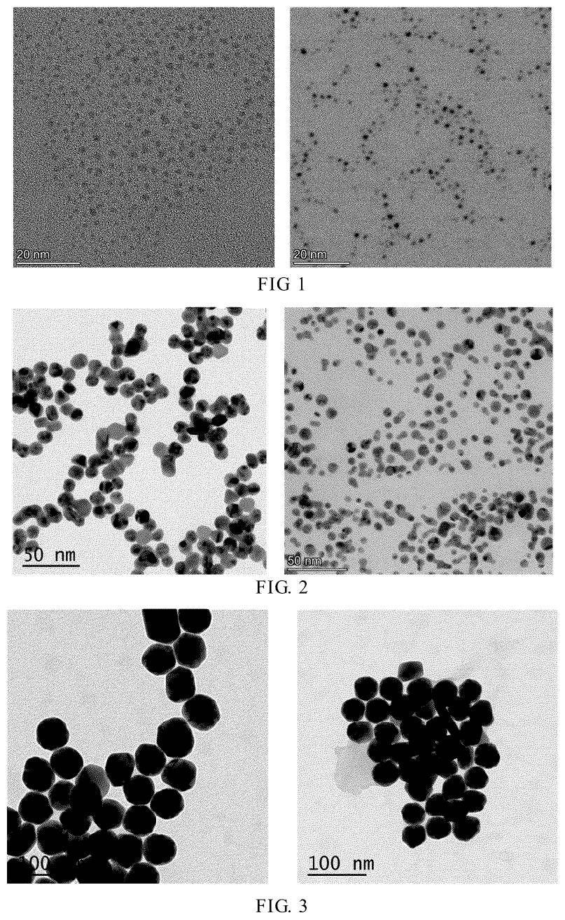

2 mL of 5 mM HAuCl₄·4H₂O solution was added to 7.85 mL of water, stirred evenly at room temperature, and 0.15 mL of 0.1 M glutathione was added dropwise and stirred continuously for 10 min. The mixture was subjected to water bath at 70° C. for 24 h in the darkness, then allowed to stand for 4 days, cooled naturally, and then centrifuged and washed with water and ethanol for 3 times each. Finally the mixture was dried in an oven at 70° C. for 12 h to obtain gold nanomaterials (3 nm), as shown in FIG. 1.

### Example 2

### Preparation of Noble Metal Gold Nanomaterials (10 nm)

1 mL of 50 mM HAuCl₄·4H₂O solution and 49 mL of water were added to a round-bottom flask, stirred and heated to boiling, then 10 mL 1% sodium citrate aqueous solution was quickly added, and continuously heated to boil for 10 min; after heating was stopped, the mixture was cooled to room temperature, then allowed to stand for 4 days, cooled naturally, and then centrifuged and washed with water and ethanol for 3 times each. Finally the mixture was dried in an oven at 70° C. for 12 h to obtain gold nanomaterials (10 nm), as shown in FIG. 2.

### Example 3

### Preparation of Noble Metal Gold Nanomaterials (40 nm)

1 mL of 50 mM HAuCl₄·4H₂O solution and 49 mL of water were added to a round-bottom flask, stirred and heated to boiling, then 10 mL 1% sodium citrate aqueous solution was quickly added, and continuously heated to boil for 10 min; after heating was stopped, the mixture was cooled to room temperature, then allowed to stand for 4 days, cooled naturally, and then centrifuged and washed with water and ethanol for 3 times each. Finally the mixture was dried in an oven at 70° C. for 12 h to obtain gold nanomaterials (40 nm), as shown in FIG. 3.

### Example 4

Preparation of Zn_{0.2}Fe_{2.8}O₄ Magnetic Nanoparticles (4 nm)

1.73 mmol of ammonium ferrous sulfate hexahydrate and 0.534 mmol of zinc sulfate heptahydrate were added to 20 mL of ultrapure water to prepare a solution. Then 1 g of sodium hydroxide was added to the mixture of oleic acid (10 mL) and ethanol (10 mL), stirred until completely dissolved, then 20 mL of ammonium ferrous sulfate and zinc sulfate solution were added; after the color of the mixture turned brownish red, the mixture was transferred to a 50-mL reactor, heated at 230° C. for 8 h; after the reactor was cooled, taken out, washed three times by centrifugation with ethanol, and dispersed in 20 mL of cyclohexane to obtain the desired oil-soluble magnetic nanoparticles. Then, 2 g of citric acid and 20 mL of oil-soluble magnetic nanoparticles were added to 30 mL of the mixture of chloroform/DMF (v/v: 1/1), stirred for 12 h, washed three times by centrifugation with ethanol, and dispersed in 20 mL of water to obtain the required water-soluble magnetic nanoparticles, then allowed to stand for 4 days, cooled naturally, and then centrifuged and washed with water and ethanol for 3 times each. Finally the mixture was dried in an oven at 70° C. for 12 h to obtain Zn_{0.2}Fe_{2.8}O₄ magnetic nanoparticles (4 nm), as shown in FIG. 4.

### Example 5

Preparation of Zn_{0.2}Fe_{2.8}O₄ Magnetic Nanoparticles (7 nm)

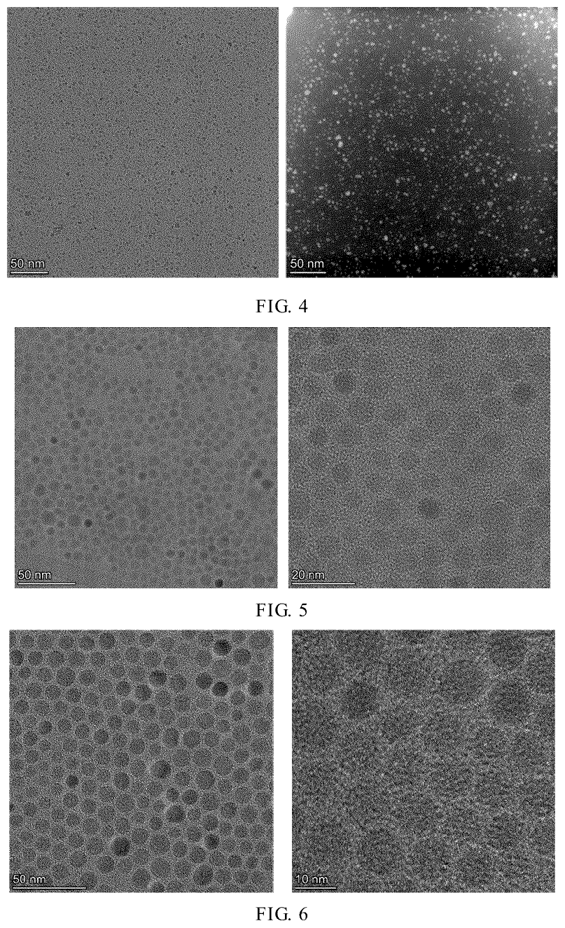

1.73 mmol of ammonium ferrous sulfate hexahydrate and 0.534 mmol of zinc sulfate heptahydrate were added to 20 mL of ultrapure water to prepare a solution. Then 1 g of sodium hydroxide was added to the mixture of oleic acid (10 mL) and ethanol (10 mL), stirred until completely dissolved, then 20 mL of ammonium ferrous sulfate and zinc sulfate solution were added; after the color of the mixture turned brownish red, the mixture was transferred to a 50-mL reactor, heated at 230° C. for 16 h; after the reactor was cooled, taken out, washed three times by centrifugation with ethanol, and dispersed in 20 mL of cyclohexane to obtain the desired oil-soluble magnetic nanoparticles. Then, 2 g of citric acid and 20 mL of oil-soluble magnetic nanoparticles were added to 30 mL of the mixture of chloroform/DMF (v/v: 1/1), stirred for 12 h, washed three times by centrifugation with ethanol, and dispersed in 20 mL of water to obtain the required water-soluble magnetic nanoparticles, then allowed to stand for 4 days, cooled naturally, and then centrifuged and washed with water and ethanol for 3 times each. Finally the mixture was dried in an oven at 70° C. for 12 h to obtain Zn_{0.2}Fe_{2.8}O₄ magnetic nanoparticles (7 nm), as shown in FIG. 5.

### Example 6

Preparation of Zn_{0.2}Fe_{2.8}O₄ Magnetic Nanoparticles (10 nm)

1.73 mmol of ammonium ferrous sulfate hexahydrate and 0.534 mmol of zinc sulfate heptahydrate were added to 20 mL of ultrapure water to prepare a solution. Then 1 g of sodium hydroxide was added to the mixture of oleic acid (10 mL) and ethanol (10 mL), stirred until completely dissolved, then 20 mL of ammonium ferrous sulfate and zinc sulfate solution were added; after the color of the mixture turned brownish red, the mixture was transferred to a 50-mL reactor, heated at 230° C. for 24 h; after the reactor was cooled, taken out, washed three times by centrifugation with ethanol, and dispersed in 20 mL of cyclohexane to obtain the desired oil-soluble magnetic nanoparticles. Then, 2 g of citric acid and 20 mL of oil-soluble magnetic nanoparticles were added to 30 mL of the mixture of chloroform/DMF (v/v: 1/1), stirred for 12 h, washed three times by centrifugation with ethanol, and dispersed in 20 mL of water to obtain the required water-soluble magnetic nanoparticles, then allowed to stand for 4 days, cooled naturally, and then centrifuged and washed with water and ethanol for 3 times each. Finally the mixture was dried in an oven at 70° C. for 12 h to obtain Zn_{0.2}Fe_{2.8}O₄ magnetic nanoparticles (7 nm), as shown in FIG. 6.

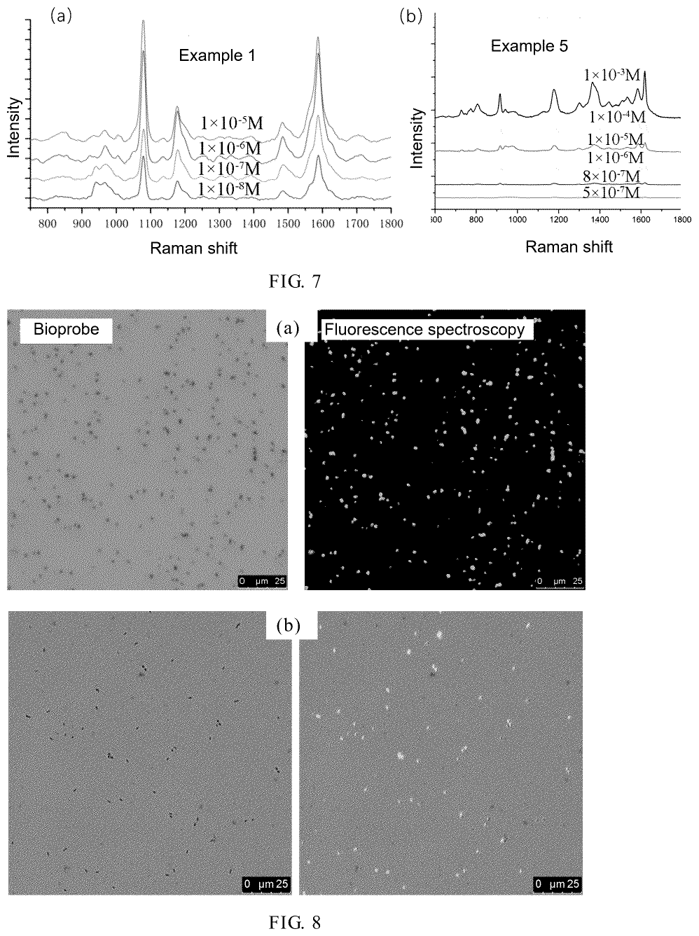

The noble metal gold nanomaterials of Example 1 and the magnetic nanoparticles of Example 5 were used as SERS substrates to perform SERS spectroscopy of different concentrations of P-carboxythiophenol (4MBA) and crystal violet molecules (CV) under the action of 633 nm excitation wavelength. The results are shown in FIG. 7. The noble metal gold nanomaterials of Example 1 had excellent SERS detection imaging performance for low concentrations of 4MBA molecules, and the magnetic nanoparticles of Example 5 had excellent SERS detection imaging performance for low concentrations of crystal violet molecules.

The luminescent indicator CCK-8 was modified on the surface of the noble metal gold nanomaterials of Example 1 and the magnetic nanoparticles of Example 5 respectively for characterization to obtain the fluorescence luminescence images of the materials. FIG. 8 (a) was a fluorescence spectrum of a luminescent indicator CCK-8 modified on the surface of magnetic nanoparticles in Example 5, and FIG. 8 (b) was a fluorescence spectrum of a luminescent indicator CCK-8 modified on the surface of noble metal gold nanomaterials in Example 1.

### Comparative Example 1

Preparation of Fe₃O₄ Magnetic Nanoparticles

2 mmol of ammonium ferrous sulfate hexahydrate was added to 20 mL of ultrapure water to prepare a solution. Then 1 g of sodium hydroxide was added to a mixture of oleic acid (10 mL) and ethanol (10 mL), stirred until completely dissolved, then 20 mL of ammonium ferrous sulfate solution was added; after the color of the mixture turned brownish red, the mixture was transferred to a 50-mL reactor, heated at 230° C. for 16 h; after the reactor was cooled, taken out, washed three times by centrifugation with ethanol, and dispersed in 20 mL of cyclohexane to obtain the desired oil-soluble magnetic nanoparticles. Then, 2 g of citric acid and 20 mL of oil-soluble magnetic nanoparticles were added to 30 mL of the mixture of chloroform/DMF (v/v: 1/1), stirred for 12 h, washed three times by centrifugation with ethanol, and dispersed in 20 mL of water to obtain the required magnetic nanoparticles, then allowed to stand for 4 days, cooled naturally, and then centrifuged and washed with water and ethanol for 3 times each. Finally the mixture was dried in an oven at 70° C. for 12 h to obtain Fe₃O₄ magnetic nanoparticles.

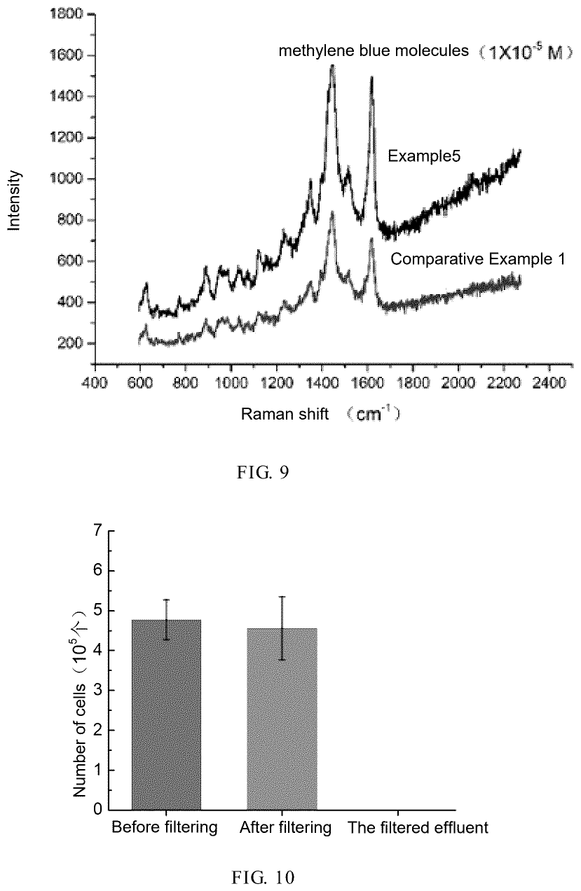

The SERS detection imaging capabilities of the magnetic nanoparticles of Example 5 and the Fe₃O₄ magnetic nanoparticles of Comparative Example 1 as SERS substrates were compared; under the action of 532 nm excitation wavelength, the SERS spectroscopy was performed on methylene blue molecules with a concentration of 1×10⁻⁵ mol/L to generate SERS spectrogram. As shown in FIG. 9, the nanoparticles doped with Zn had a better SERS enhancement effect; possibly after Zn doping, more orbital energy levels would be provided, which was conducive to the charge transfer effect between materials and molecules, thereby enhancing the SERS effect. The magnetic semiconductor SERS bioprobe formed based on Zn_{0.2}Fe_{2.8}O₄ magnetic nanoparticles and the noble metal SERS bioprobe were used in combination for in-vitro testing, which had higher testing sensitivity and better accuracy.

### Example 7

### Preparation of Au-IR783-rBSA-Trop2 Antibody Protein Nanoprobe

### (1) Preparation of Noble Metal Gold Nanomaterials

2 mL of 5 mM HAuCl₄. 4H₂O solution was added to 7.85 mL of water, stirred evenly at room temperature, and 0.15 mL of 0.1 M glutathione was added dropwise and stirred continuously for 10 min. The mixture was subjected to water bath at 70° C. for 24 h in the darkness, then allowed to stand for 4 days, cooled naturally, and then centrifuged and washed with water and ethanol for 3 times each. Finally the mixture was dried in an oven at 70° C. for 12 h to obtain gold nanomaterials (3 nm).

### (2) Preparation of Au-IR783 Nanoparticles

1 mg of gold nanomaterial was added to 15 mL of 0.05 mmol/L IR783 ethanol solution, stirred with a PTFE rod for 2 h, thoroughly washed with deionized water, and finally dispersed in 18 mL of deionized water to obtain Au-IR783 solution.

### (3) Preparation of Au-IR783-rBSA Nanoparticles

18 mL of Au-IR783 solution, 8 mL of CH₃CH₂OH and 600 μL of NH₃·H₂O were mixed and stirred with a PTFE rod for 20 min, and then 2 mL of rBSA solution (40 mg/mL) was slowly added slowly; 5 hours later, the mixture was thoroughly washed with deionized water, and dispersed in 8 mL of deionized water to obtain Zn_{0.2}Fe_{2.8}O₄-IR780-rBSA solution.

### (4) Preparation of Au-IR783-rBSA-Trop2 Antibody Protein Nanoparticles

4 mL of Au-IR783-rBSA solution was adsorbed with a magnet to obtain Au-IR783-rBSA nanoparticles; the supernatant was discarded, then 4 mL of Tris-HCl solution (10 mM, pH=8.5) was added, and then 40 μg of Trop2 antibody protein was added, stirred at room temperature for 12 h, washed with PBS three times, and finally dispersed in 4 mL of PBS solution to obtain Au-IR783-rBSA-Trop2 antibody protein bioprobe.

### Example 8

Preparation of Zn_{0.2}Fe_{2.8}O₄-Alizarin Red-PDA-Trop2 Antibody Protein Nanoprobe
(1) Preparation of Zn_{0.2}Fe_{2.8}O₄ Magnetic Nanoparticles

1.73 mmol of ammonium ferrous sulfate hexahydrate and 0.534 mmol of zinc sulfate heptahydrate were added to 20 mL of ultrapure water to prepare a solution. Then 1 g of sodium hydroxide was added to the mixture of oleic acid (10 mL) and ethanol (10 mL), stirred until completely dissolved, then 20 mL of ammonium ferrous sulfate and zinc sulfate solution were added; after the color of the mixture turned brownish red, the mixture was transferred to a 50-mL reactor, heated at 230° C. for 16 h; after the reactor was cooled, taken out, washed three times by centrifugation with ethanol, and dispersed in 20 mL of cyclohexane to obtain the desired oil-soluble magnetic nanoparticles. Then, 2 g of citric acid and 20 mL of oil-soluble magnetic nanoparticles were added to 30 mL of the mixture of chloroform/DMF (v/v: 1/1), stirred for 12 h, washed three times by centrifugation with ethanol, and dispersed in 200 ml of water to obtain a Zn_{0.2}Fe_{2.8}O₄ magnetic nanoparticle solution.

(2) Preparation of Zn_{0.2}Fe_{2.8}O₄-Alizarin Red Nanoparticles

150 μL of 1 mmol/L alizarin red ethanol solution was added to 15 mL of magnetic nanoparticle solution (solvent: water, concentration: 0.21 mg/mL), stirred with a PTFE rod for 2 h, thoroughly washed with deionized water, and finally dispersed in 18 mL of deionized water to obtain a Zn_{0.2}Fe_{2.8}O₄-alizarin red solution.

(3) Preparation of Zn_{0.2}Fe_{2.8}O₄-Alizarin Red-PDA Nanoparticles

18 mL of Zn_{0.2}Fe_{2.8}O₄-alizarin red solution (solvent: water, concentration: 0.19 mg/mL), 8 mL of CH₃CH₂OH and 600 μL of NH₃·H₂O were mixed and stirred with a PTFE rod for 20 min. Then, 2 mL of polydopamine solution (40 mg/mL) was slowly added; 5 hours later, the mixture was thoroughly washed with deionized water, and dispersed in 8 mL of deionized water to obtain Zn_{0.2}Fe_{2.8}O₄-alizarin red-PDA nanoparticle solution.

(4) Preparation of Zn_{0.2}Fe_{2.8}O₄-Alizarin Red-PDA-Trop2 Antibody Protein Nanoparticles

4 mL of Zn_{0.2}Fe_{2.8}O₄-alizarin red-PDA-nanoparticle solution was adsorbed with a magnet to obtain Zn_{0.2}Fe_{2.8}O₄-alizarin red-PDA nanoparticles; the supernatant was discarded, then 4 mL of Tris-HCl solution (10 mM, pH=8.5) was added, and then 40 μg of Trop2 antibody protein was added, stirred at room temperature for 12 h, washed with PBS three times, and finally dispersed in 4 mL of PBS solution to obtain Zn_{0.2}Fe_{2.8}O₄-alizarin red-PDA-40 μg Trop2 antibody protein solution.

The Zn_{0.2}Fe_{2.8}O₄-alizarin red-PDA-40 μg Trop2 antibody protein nanoprobe of Example 8 was co-incubated with tumor cells, and magnetically enriched in the magnetic enrichment module of the circulating tumor cell detection equipment. The enrichment effect is shown in FIG. 10. Tumor cells had excellent binding properties with the Zn_{0.2}Fe_{2.8}O₄ nanoprobe. After magnetic enrichment, tumor cells were enriched, and there were no tumor cells in the filtered effluent, indicating that Zn_{0.2}Fe_{2.8}O₄ nanoprobe had good ability to enrich and capture tumor cells.

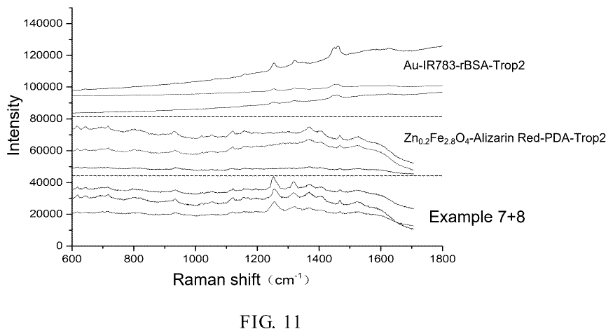

The Au-IR783-rBSA-Trop2 antibody protein nanoprobe of Example 7 was co-incubated with MCF7 breast cancer cells alone, then SERS spectroscopy was performed. The SERS spectrum is shown in FIG. 11; the Zn_{0.2}Fe_{2.8}O₄-alizarin red-PDA-Trop2 antibody protein nanoprobe of Example 8 was co-incubated with MCF7 breast cancer cells alone, and then SERS spectroscopy was performed. The SERS spectrum is shown in FIG. 11; the Au-IR783-rBSA-Trop2 antibody protein nanoprobe of Example 7 and the Zn_{0.2}Fe_{2.8}O₄-alizarin red-PDA-Trop2 antibody protein nanoprobe of Example 8 were co-incubated with MCF7 breast cancer cells, magnetically enriched and separated, and then SERS spectroscopy was performed. The SERS spectrum is shown in FIG. 11.

As shown in FIG. 11, when only a single probe was used to detect cancer cells, the SERS signal peaks were few, unstable, and had poor uniformity; when Au-IR783-rBSA-Trop2 antibody protein nanoprobe and Zn_{0.2}Fe_{2.8}O₄-alizarin red-PDA-Trop2 antibody protein nanoprobe of Example 8 were used to jointly detect cancer cells, there were many SERS signal peaks, and the signals were stable, which was conducive to improving the detection sensitivity and accuracy.

The various aspects, embodiments, and features of the present disclosure should be considered to be illustrative in all aspects and not limiting of the present disclosure, and the scope of the present disclosure is only defined by the appended claims. Without departing from the claimed spirit and scope of the present disclosure, other embodiments, modifications, and uses will be apparent to those skilled in the art.

In the preparation method of the present disclosure, the order of the steps is not limited to the order listed, and for those skilled in the art, the order of the steps is also within the scope of protection of the present disclosure without creative work. In addition, two or more steps or actions may be performed simultaneously.

Finally, it should be noted that the specific embodiments described herein are merely examples of the present disclosure, and are not intended to limit the implementation methods of the present disclosure. Those skilled in the art of the present disclosure may make various modifications or supplements to the specific embodiments described or replace them in a similar manner, and it is not necessary and impossible to provide all examples of all implementation methods herein. However, these obvious changes or modifications derived from the essential spirit of the present disclosure will still fall within the scope of protection of the present disclosure, and will not be interpreted as any additional restriction; otherwise, it is contrary to the spirit of the present disclosure.

1. A multifunctional composite bioprobe, comprising one or more of noble metal SERS bioprobes and one or more of magnetic semiconductor SERS bioprobes;
the noble metal SERS bioprobe comprises a noble metal material with SERS performance, the noble metal material comprises one or more of gold, silver, palladium, and copper, or comprises a composite material containing one or more of gold, silver, palladium, and copper;
the magnetic semiconductor SERS bioprobe comprises a magnetic metal oxide with SERS performance, and the magnetic metal oxide comprises an oxide material containing one or more of Fe, Zn, Co, Ni, Cr, and Mn.

2. The multifunctional composite bioprobe according to claim 1, wherein the particle size of the noble metal SERS bioprobe is 0.1 nm-10,000 nm, and the particle size of the magnetic semiconductor SERS bioprobe is 0.1 nm-10,000 nm.

3. (canceled)

4. The multifunctional composite bioprobe according to claim 1, wherein the magnetic metal oxide is an oxide material formed by Fe and one or more of Zn, Co, Ni, Cr, and Mn.

5. The multifunctional composite bioprobe according to claim 4, wherein the magnetic metal oxide is ZnₓFe₃₋ₓO₄, 0<x<3.

6. The multifunctional composite bioprobe according to claim 4, wherein a method for preparing the magnetic metal oxide comprises the following steps: mixing a solution of one or more of a zinc salt, a cobalt salt, a nickel salt, a chromium salt, a manganese salt and an iron salt with a solution formed by an alkaline inorganic matter, an organic acid with long alkyl chain and a polar organic solvent, reacting to obtain initial magnetic particles, and performing phase transfer to obtain magnetic metal oxide nanoparticles.

7. The multifunctional composite bioprobe according to claim 6, wherein iron salts comprise one or more of ferric chloride, ferrous chloride, ferric sulfate, ferrous sulfate, ammonium ferrous sulfate, ferric nitrate, and ferrous nitrate; zinc salts comprise one or more of zinc chloride, zinc sulfate, and zinc nitrate; cobalt salts comprise one or more of cobalt chloride, cobalt sulfate, and cobalt nitrate; nickel salts comprise one or more of nickel chloride, nickel sulfate, and nickel nitrate; chromium salts comprise one or more of chromium chloride, chromium sulfate, and chromium nitrate; manganese salts comprise one or more of manganese chloride, manganese sulfate, and manganese nitrate;
the alkaline inorganic matter is one or more of barium hydroxide, potassium hydroxide, calcium hydroxide, sodium hydroxide and ammonium hydroxide;
the organic acid with long alkyl chain is one or more of oleic acid, octadecanoic acid, hexadecanoic acid, tetradecanoic acid and dodecanoic acid.

8. The multifunctional composite bioprobe according to claim 6, wherein the phase transfer comprises the following steps: adding the initial magnetic particles and citric acid to the organic solvent, and stirring at room temperature for 1-50 h.

9. The multifunctional composite bioprobe according to claim 1, wherein the noble metal SERS bioprobe comprises a noble metal material, Raman/fluorescence signal molecules, biomacromolecules and antibody targeting proteins in sequence from the inside to the outside; and the magnetic semiconductor SERS bioprobe comprises a magnetic metal oxide, Raman/fluorescence signal molecules, biomacromolecules and antibody targeting proteins in sequence from the inside to the outside.

10. A method for preparing the multifunctional composite bioprobe of claim 1, comprising the following steps: mixing one or more of the noble metal SERS bioprobes with one or more of the magnetic semiconductor SERS bioprobes in a liquid or solid form to obtain the multifunctional composite bioprobe.

11. Use of the multifunctional composite bioprobe of claim 1 in in-vitro testing, comprising the following steps: mixing one or more of the noble metal SERS bioprobes with one or more of the magnetic semiconductor SERS bioprobes in a liquid or solid form and then adding the mixture to a system to be tested; or adding one or more of the noble metal SERS bioprobes and one or more of the magnetic semiconductor SERS bioprobes to a system to be tested successively in a liquid or solid form.

12. The use of the multifunctional composite bioprobe of claim 11 in in-vitro testing, further comprising the following steps: after adding the multifunctional composite bioprobe to the system to be tested and combining the multifunctional composite bioprobe with the target in the system to be tested, carrying out magnetic enrichment and separation, then performing Raman spectroscopy and/or fluorescence spectroscopy to determine the concentration of the target in the system to be tested.

13. An in vitro detection device, comprising the multifunctional composite bioprobe of claim 1.

14. The multifunctional composite bioprobe according to claim 1, wherein the particle size of the noble metal material is 1-100 nm; the particle size of the magnetic metal oxide is 1-50 nm.

15. The multifunctional composite bioprobe according to claim 6, wherein the reaction temperature is 200-500° C., and the reaction time is 2-50 h.

16. The multifunctional composite bioprobe according to claim 6, wherein the organic solvent is chloroform, N,N-dimethylformamide, trichloromethane, carbon tetrachloride, formamide, DMSO, tetrahydrofuran or pyridine.
